(19) United States
(12) Patent Application Publication
(10) Pub. No.: US 2025/0195260 A1
(43) Pub. Date: Jun. 19, 2025

(54) FLUID COLLECTION ASSEMBLIES INCLUDING AT LEAST ONE SECUREMENT BODY

(71) Applicant: PUREWICK CORPORATION, Covington, GA (US)

(72) Inventors: Ashley Marie Johannes, Statham, GA (US); Shahab Siddiqui, Lawrenceville, GA (US); Kathleen Davis, Atlanta, GA (US)

(21) Appl. No.: 19/069,480

(22) Filed: Mar. 4, 2025

Related U.S. Application Data
(63) Continuation of application No. 17/646,771, filed on Jan. 3, 2022, now Pat. No. 12,268,627.
(60) Provisional application No. 63/134,450, filed on Jan. 6, 2021.

(51) Int. Cl. A61F 5/451; A61F 5/44

(52) U.S. Cl. CPC A61F 5/451; A61F 5/4404

(57) ABSTRACT

Embodiments disclosed herein include fluid collection assemblies with at least one securement body. In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber and at least one securement body configured to limit movement of the fluid collection assembly relative to a region about a urethral opening of a patient. The at least one securement body includes at least one of a plurality of fibers exhibiting an average lateral dimension of about 5 μm or less, a plurality of suction cups, or at least one friction material exhibiting a coefficient of static friction that is greater than at least a portion of the at least one porous material.

### CROSS-REFERENCE TO RELATED APPLICATIONS

This application is a continuation of U.S. patent application Ser. No. 17/646,771 filed on Jan. 3, 2022, which claims priority to U.S. Provisional Patent Application No. 63/134,450 filed on Jan. 6, 2021, the disclosure of each of which is incorporated herein, in its entirety, by this reference.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection assemblies continue to seek new and improved devices, systems, and methods to collect urine.

### SUMMARY

Embodiments disclosed herein include fluid collection assemblies with at least one securement body, fluid collection systems including the same, and methods of using the same. In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber and at least one securement body configured to limit movement of the fluid collection assembly relative to a region about a urethral opening of a patient. The at least one securement body includes at least one of a plurality of fibers exhibiting an average lateral dimension of about 5 μm or less, a plurality of suction cups, or at least one friction material exhibiting a coefficient of static friction that is greater than at least a portion of the at least one porous material.

In an embodiment, a fluid collection system is disclosed. The fluid collection system includes a fluid storage container configured to hold one or more bodily fluids therein. The fluid collection system also includes a fluid collection assembly. The fluid collection assembly includes a fluid impermeable barrier at least defining a chamber, at least one opening, and a fluid outlet. The fluid collection assembly also includes at least one porous material disposed in the chamber and at least one securement body configured to limit movement of the fluid collection assembly relative to a region about a urethral opening of a patient. The at least one securement body includes at least one of a plurality of fibers exhibiting an average lateral dimension of about 5 μm or less, a plurality of suction cups, or at least one friction material exhibiting a coefficient of static friction that is greater than at least a portion of the at least one porous material. The fluid collection system further includes a vacuum source in fluid communication with the fluid storage container and the fluid collection assembly. The vacuum source is configured to draw the one or more bodily fluids from the fluid collection assembly and deposit the one or more bodily fluids in the fluid storage container via one or more conduits.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIG. 1B is a cross-sectional view of the fluid collection assembly taken along plane 1B-1B shown in FIG. 1A, according to an embodiment.

FIG. 1C is an enlarged cross-sectional schematic of a portion of the base, according to an embodiment.

FIGS. 1D to 1F are cross-sectional schematics of different bases that may be used with the fluid collection assembly, according to different embodiments.

FIG. 2 is a cross-sectional schematic of a fluid collection assembly, according to an embodiment.

FIG. 3A is an isometric view of a fluid collection assembly, according to an embodiment.

FIG. 3B is a cross-sectional schematic of the fluid collection assembly taken along plane 3B-3B shown in FIG. 3A, according to an embodiment.

FIG. 4A is an isometric view of a fluid collection assembly that includes at least one friction material, according to an embodiment.

FIG. 4B is a cross-sectional schematic of the fluid collection assembly taken along plane 4B-4B shown in FIG. 4A, according to an embodiment.

FIGS. 4C-4E are isometric views of different fluid collection assemblies that include the friction material disposed therein in different arrangements, according to different embodiments.

FIG. 5A is a cross-sectional schematic of a portion of a fluid collection assembly, according to an embodiment.

FIG. 5B is a cross-sectional schematic of a portion of a fluid collection assembly, according to an embodiment.

FIG. 6 is a block diagram of a system for fluid collection, according to an embodiment.

### DETAILED DESCRIPTION

Embodiments disclosed herein include fluid collection assemblies with at least one securement body, fluid collection systems including the same, and methods of using the same. An example fluid collection assembly may include a male fluid collection assembly configured to receive one or more bodily fluids (e.g., urine, blood, sweat, etc.) from a male urethral opening or a female fluid collection assembly configured to receive the bodily fluids from a female urethral opening. The fluid collection assembly includes a fluid impermeable barrier that at least defines a chamber, at least one opening, and a fluid outlet. The fluid collection assembly also includes at least one porous material disposed in the chamber and at least one securement body. The securement body is disposed on one or more components of the fluid collection assembly and is configured to limit movement of the fluid collection assembly relative to a region about a urethral opening of the patient. For example, the securement body may be disposed on one or more of at least a portion of the fluid impermeable barrier, at least a portion of the porous material that extends across the opening, or a base. Examples of securement bodies include a plurality of fibers extending from at least one exterior surface of the fluid collection assembly, a plurality of suction cups, or a friction material that exhibits a coefficient of friction that is greater than at least a portion of the porous material.

During use, the fluid collection assembly is positioned adjacent to or receives the urethral opening of the patient. The patient may discharge one or more bodily fluids and the fluid collection assembly may receive the bodily fluids. However, movement of the fluid collection assembly may prevent the fluid collection assembly from receiving all or substantially all of the bodily fluids that are discharged by the patient. For example, movement of the fluid collection assembly may cause bodily fluids to leak. The leaked bodily fluids may create patient discomfort by causing the skin of the patient to remain moist, creating unsanitary conditions, requiring cleaning of the patient, and causing embarrassment to the patient.

Some conventional fluid collection assemblies rely on chemical adhesives or contact between the thighs of the patient and the fluid impermeable barrier to maintain the position of the fluid collection assembly and prevent bodily fluid leaks. However, the chemical adhesives may be painful to remove from the patient since such chemical adhesives may be attached to hair and sensitive regions of the patient (e.g., the region about the urethral opening). Further, the contact between the thighs of the patient and the fluid impermeable barrier may be unable to maintain the position of the fluid collection assembly because the patient is too thin (e.g., has gaps between the thighs that prevent contact with the fluid impermeable barrier) or the patient moves. The fluid collection assemblies that include at least one securement body (e.g., the plurality of fibers, the friction material, and/or the plurality of suction cups) are improvements over such conventional fluid collection assemblies. In an example, the securement body may engage the patient to limit movement of the fluid collection assembly but, unlike chemical adhesives, the securement body may be easily removed when certain forces are applied thereto. Further, the securement body may maintain the position of the fluid collection assembly even when the patient is too thin for the thighs of the patient to contact the fluid collection assembly and/or when the patient moves.

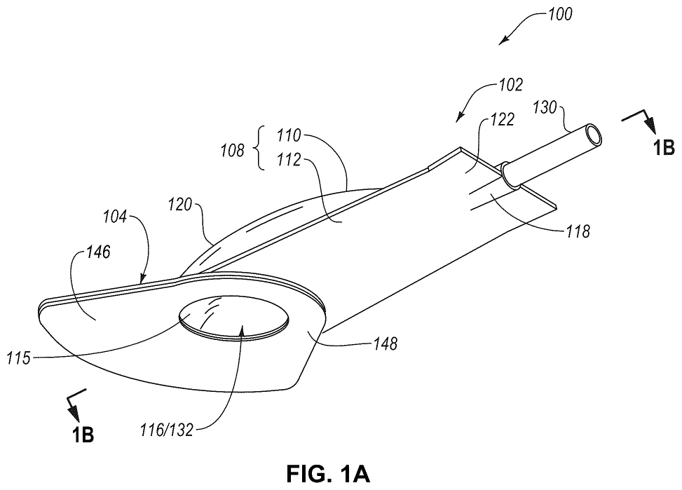

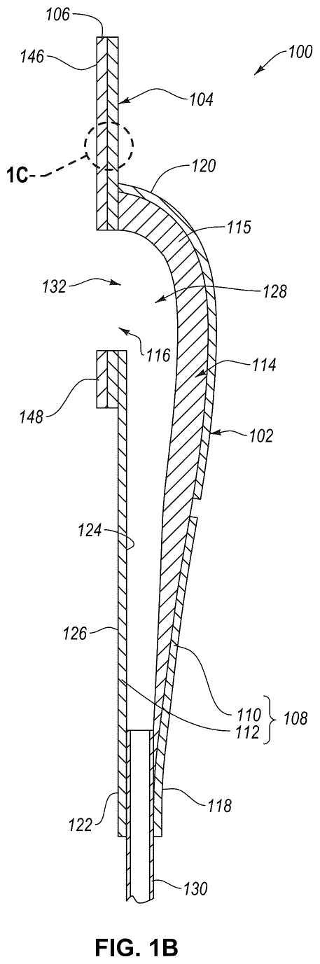

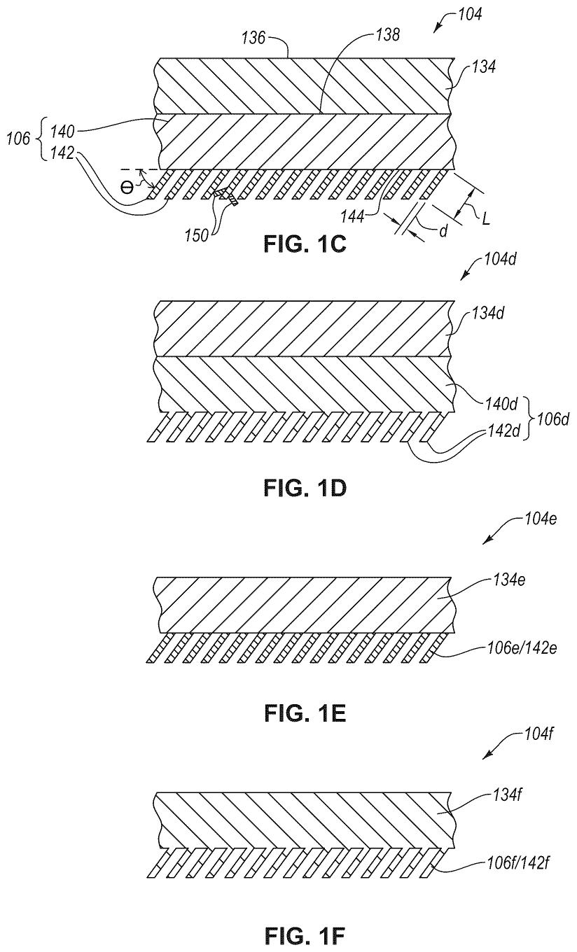

FIG. 1A is an isometric view of a fluid collection assembly 100, according to an embodiment. FIG. 1B is a cross-sectional view of the fluid collection assembly 100 taken along plane 1B-1B shown in FIG. 1A, according to an embodiment. The fluid collection assembly 100 is an example of a male fluid collection assembly though, in some embodiments, the fluid collection assembly 100 may be used to receive bodily fluids from a female urethral opening. The fluid collection assembly 100 includes a sheath 102 and a base 104. The base 104 is configured to be attached (e.g., permanently attached or configured to be permanently attached) to the sheath 102. The base 104 is also configured to be attached to the region about the urethral opening (e.g., penis) of the patient. The base 104 includes at least one securement body 106.

The sheath 102 includes a fluid impermeable barrier 108 that is at least partially formed from a first panel 110 and a second panel 112. The first panel 110 and the second panel 112 may be attached or integrally formed together (e.g., exhibits single piece construction). In an embodiment, as illustrated, the first panel 110 and the second panel 112 are distinct sheets. The fluid impermeable barrier 108 also defines a chamber 114 between the first panel 110 and the second panel 112, an opening 116 at a first end region 120 of the sheath 102, and an fluid outlet 118 at a second end region 122 of the sheath 102. The sheath 102 also includes at least one porous material 115 disposed in the chamber 114.

The inner surface(s) 124 of the fluid impermeable barrier 108 (e.g., inner surfaces of the first and second panels 110, 112) at least partially defines the chamber 114 within the fluid collection assembly 100. The fluid impermeable barrier 108 temporarily stores the bodily fluids in the chamber 114. The fluid impermeable barrier 108 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, or combinations thereof. As such, the fluid impermeable barrier 108 substantially prevents the bodily fluids from passing through the fluid impermeable barrier 108. In an example, the fluid impermeable barrier 108 may be air permeable and fluid impermeable. In such an example, the fluid impermeable barrier 108 may be formed of a hydrophobic material that defines a plurality of pores. At least one or more portions of at least an outer surface 126 of the fluid impermeable barrier 108 may be formed from a soft and/or smooth material, thereby reducing chaffing.

In an embodiment, at least one of the first panel 110 or the second panel 112 is formed from an at least partially transparent fluid impermeable material, such as polyethylene, polypropylene, polycarbonate, or polyvinyl chloride. Forming at least one of the first panel 110 or the second panel 112 from an at least partially transparent fluid impermeable material allows a person (e.g., medical practitioner) to examiner the penis. In some embodiments, both the first panel 110 and the second panel 112 are formed from at least partially transparent fluid impermeable material. Selecting at least one of the first panel 110 or the second panel 112 to be formed from an at least partially transparent impermeable material allows the penis to be examined without detaching the entire fluid collection assembly 100 from the region about the penis. For example, the chamber 114 may include a penis receiving area 128 that is configured to receive the penis of the individual when the penis extends into the chamber 114. The penis receiving area 128 may be defined by at least the porous material 115 and at least a portion of the at least partially transparent material of the first panel 110 and/or the second panel 112. In other words, the porous material 115 is positioned in the chamber 114 such that the porous material 115 is not positioned between the penis and at least a portion of the transparent portion of the first panel 110 and/or second panel 112 when the penis is inserted into the chamber 114 through the opening 116. The porous material 115 is generally not transparent and, thus, the portion of the at least partially transparent material of the first panel 110 and/or the second panel 112 that defines the penis receiving area 128 forms a window which allows the person to view into the penis receiving area 128 and examine the penis.

The opening 116 defined by the fluid impermeable barrier 108 provides an ingress route for fluids to enter the chamber 114 when the penis is a buried penis and allow the penis to enter the chamber 114 (e.g., the penis receiving area 128) when the penis is not buried. The opening 116 may be defined by the fluid impermeable barrier 108 (e.g., an inner edge of the fluid impermeable barrier 108). For example, the opening 116 is formed in and extends through the fluid impermeable barrier 108 thereby enabling bodily fluids to enter the chamber 114 from outside of the fluid collection assembly 100.

The fluid impermeable barrier 108 defines an fluid outlet 118 sized to receive an conduit 130. The conduit 130 may be at least partially disposed in the chamber 114 or otherwise in fluid communication with the chamber 114 through the fluid outlet 118. The fluid outlet 118 may be sized and shaped to form an at least substantially fluid tight seal against the conduit 130 thereby substantially preventing the bodily fluids from escaping the chamber 114. In an embodiment, the fluid outlet 118 may be formed from a portion of the first panel 110 and the second panel 112 that are not attached or integrally formed together. In such an embodiment, the fluid impermeable barrier 108 may not include a cap exhibiting a rigidity that is greater than the portions of the fluid impermeable barrier 108 thereabout which may facilitate manufacturing of the fluid collection assembly 100 may decreasing the number of parts that are used to form the fluid collection assembly 100 and may decrease the time required to manufacture the fluid collection assembly 100. The lack of the cap may make securing the conduit 130 to the fluid outlet 118 using interference fit to be difficult though, it is noted, attaching the conduit 130 to the fluid outlet 118 may still be possible. As such, the conduit 130 may be attached to the fluid outlet 118 (e.g., to the first and second panels 110, 112) using an adhesive, a weld, or otherwise bonding the fluid outlet 118 to the fluid outlet 118. Attaching the conduit 130 to the fluid outlet 118 may prevent leaks and may prevent the conduit 130 from inadvertently becoming detached from the fluid outlet 118. In an example, the conduit 130 may be attached to the fluid outlet 118 in the same manufacturing step that attaches the first and second panels 110, 112 together.

As previously discussed, the sheath 102 includes at least one porous material 115 disclosed in the chamber 114. The porous material 115 may direct the bodily fluids to one or more selected regions of the chamber 114, such as away from the penis and towards the fluid outlet 118. In an embodiment, the porous material 115 includes a fluid permeable membrane extending across the opening 116 and a fluid permeable support since the fluid permeable membrane may be formed from a relatively foldable, flimsy, or otherwise easily deformable material. For example, the fluid permeable support may be positioned such that the fluid permeable membrane is disposed between the fluid permeable support and the fluid impermeable barrier 108. As such, the fluid permeable support may support and maintain the position of the fluid permeable membrane. The fluid permeable membrane may include fabric, such as a gauze (e.g., a silk, linen, or cotton gauze), another soft fabric, or another smooth fabric. The fluid permeable support may include any of the fluid permeable membrane materials disclosed above in a more dense or rigid form, a porous polymer (e.g., nylon, polyester, polyurethane, polyethylene, polypropylene, etc.) structure or an open cell foam, spun nylon fiber, a natural material (e.g., cotton, wool, silk, or combinations thereof), any other suitable material, or combinations thereof. In an embodiment, the porous material 115 may only include one of the fluid permeable membrane or the fluid permeable support. In an embodiment, the porous material 115 includes a first porous layer, a second porous layer, and a plurality of fibers forming a layer between the first and second layers.

In an embodiment, the porous material 115 may be configured to wick any bodily fluids away from the opening 116, thereby preventing the bodily fluids from escaping the chamber 114. The permeable properties referred to herein may be wicking, capillary action, diffusion, or other similar properties or processes, and are referred to herein as “permeable” and/or “wicking.” Such “wicking” may not include absorption of the bodily fluids into the wicking material. Put another way, substantially no absorption of the bodily fluids into the material may take place after the material is exposed to the bodily fluids and removed from the bodily fluids for a time. While no absorption is desired, the term “substantially no absorption” may allow for nominal amounts of absorption of the bodily fluids into the wicking material (e.g., absorbency), such as less than about 30 wt % of the dry weight of the porous material, less than 20 wt %, less than 15 wt %, less than 10 wt %, less than about 7 wt %, less than about 5 wt %, less than about 3 wt %, less than about 2 wt %, less than about 1 wt %, or less than about 0.5 wt % of the dry weight of the porous material. The wicking material may also wick the bodily fluids generally towards an interior of the chamber 114, as discussed in more detail below. In an embodiment, the porous material 115 may include at least one absorbent or adsorbent material.

In an embodiment, the porous material 115 may be a sheet. Forming the porous material 115 as a sheet may facilitate the manufacturing of the fluid collection assembly 100. For example, forming the porous material 115 as a sheet allows the first panel 110, the second panel 112, and the porous material 115 to each be sheets. During the manufacturing of the fluid collection assembly 100, the first panel 110, the second panel 112, and the porous material 115 may be stacked and then attached to each other in the same manufacturing step. For instance, the porous material 115 may exhibit a shape that is the same size or, more preferably, slightly smaller than the size of the first panel 110 and the second panel 112. As such, attaching the first panel 110 and the second panel 112 together along the outer edges thereof may also attach the porous material 115 to the first panel 110 and the second panel 112. The porous material 115 may be slightly smaller than the first panel 110 and the second panel 112 such that the first panel 110 and/or the second panel 112 extend around the porous material 115 such that the porous material 115 does not form a passageway through the fluid impermeable barrier 108 through which the bodily fluids may leak. Also, attaching the porous material 115 to the first panel 110 and/or the second panel 112 may prevent the porous material 115 from significantly moving in the chamber 114, such as preventing the porous material 115 from bunching together near the fluid outlet 118. In an example, the porous material 115 may be attached to the first panel 110 or the second panel 112 (e.g., via an adhesive) before or after attaching the first panel 110 to the second panel 112. In an example, the porous material 115 may merely be disposed in the chamber 114 without attaching the porous material 115 to at least one of the first panel 110 or the second panel 112. In an embodiment, as will be discussed in more detail below, the porous material 115 may exhibit shapes other than a sheet, such as a hollow generally cylindrical shape.

Generally, the sheath 102 is substantially flat when the penis is not in the penis receiving area 128 and the sheath 102 is resting on a flat surface. The sheath 102 is substantially flat because the fluid impermeable barrier 108 is formed from the first panel 110 and the second panel 112 instead of a generally tubular fluid impermeable barrier. Further, as previously discussed, the porous material 115 may be a sheet, which also causes the sheath 102 to be substantially flat. The sheath 102 may also be substantially flat because the fluid collection assembly 100 may not include relatively rigid rings or caps that exhibit a rigidity that is greater than the portions of the fluid impermeable barrier 108 thereabout since such rings and caps may inhibit the sheath 102 being substantially flat. It is noted that the sheath 102 is described as being substantially flat because at least one of the porous material 115 may cause a slight bulge to form in the sheath 102 depending on the thickness of the porous material 115, the fluid outlet 118 and/or conduit 130 may cause a bulge thereabout, or the base 104 may pull on portions of the sheath 102 thereabout. It is also noted that the sheath 102 may also be compliant and, as such, the sheath 102 may not be substantially flat during use since, during use, the sheath 102 may rest on a non-flat surface (e.g., may rest on the testicles, the perineum, and/or between the thighs) and the sheath 102 may conform to the surface of these shapes.

The ability of the sheath 102 to be substantially flat when the penis is not in the penis receiving area 128 and the sheath 102 is resting on a flat surface allows the fluid collection assembly 100 to be used with a buried and a non-buried penis. For example, when the fluid collection assembly 100 is being used with a buried penis, the penis does not extend into the penis receiving area 128 which causes the sheath 102 to lie relatively flat across the aperture 132 of the base 104. When the sheath 102 lies relatively flat across the aperture 132, the porous material 115 extends across the opening 116 and the aperature 132 and is in close proximity to the buried penis. As such, the porous material 115 prevents or inhibits pooling of bodily fluids discharged from the buried penis against the skin of the individual since the porous material 115 will receive and remove at least a significant portion of the bodily fluids that would otherwise pool against the skin of the individual. Thus, the skin of the individual remains dry thereby improving comfort of using the fluid collection assembly 100 and preventing skin degradation. However, unlike other conventional fluid collection assemblies that are configured to be used with buried penises, the fluid collection assembly 100 may still be used with a non-buried penis since the non-buried penis can still be received into the penis receiving area 128, even when the penis is fully erect. Additionally, the ability of the sheath 102 to be substantially flat allows the fluid collection assembly 100 to be used more discretely than if the sheath 102 was not substantially flat thereby avoiding possibly embarrassing scenarios.

When the sheath 102 is substantially flat, the porous material 115 occupies substantially all of the chamber 114 and the penis receiving area 128 is collapsed (shown as being non-collapsed in FIG. 1B for illustrative purposes to show the penis receiving area 128). In other words, the sheath 102 may not define an region that is constantly unoccupied by the porous material 115. When the porous material 115 occupies substantially all of the chamber 114, the bodily fluids discharged into the chamber 114 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 110, the second panel 112, and the porous material 115 may be selected to be relatively flexible. The first panel 110, the second panel 112, and the porous material 115 are relatively flexible when the first panel 110, the second panel 112, and the porous material 115, respectively, are unable to maintain their shape when unsupported. The flexibility of the first panel 110, the second panel 112, and the porous material 115 may allow the sheath 102 to be substantially flat, as discussed above. The flexibility of the first panel 110, the second panel 112, and the porous material 115 may also allow the sheath 102 to conform to the shape of the penis even when the size and shape of the penis changes (e.g., becomes erect) and to minimize any unoccupied spaces in the chamber 114 in which bodily fluids may pool.

As previously discussed, the fluid collection assembly 100 includes a base 104 that is configured to be attached to the sheath 102. For example, the base 104 is configured to be permanently attached to the sheath 102. The base 104 is configured to be permanently attached to the sheath 102 when, for example, when the fluid collection assembly 100 is provided with the base 104 permanently attached to the sheath 102 or the base 104 is provided without being permanently attached to the sheath 102 but is configured to be permanently attached to the sheath 102 at some point in the future. Permanently attached means that the sheath 102 cannot be detached from the base 104 without damaging at least one of the sheath 102 or the base 104, using a blade to separate the sheath 102 from the base 104, and/or using chemicals to dissolve the adhesive that attaches the sheath 102 from the base 104. The base 104 may be permanently attached to the sheath 102 using an adhesive, sewing, heat sealing, RF welding, or US welding. In an embodiment, the base 104 is configured to be reversibly attached to the sheath 102.

As previously discussed, the base 104 includes an aperture 132. The base 104 is permanently attached to the first end region 120 of the sheath 102 such that the aperture 132 is aligned with the opening 116.

The base 104 is sized, shaped, and made of a material to be coupled to the skin that surrounds the penis (e.g., mons pubis, thighs, testicles, and/or perineum) and have the penis disposed therethrough. For example, the base 104 may define an aperture 132 configured to have the penis positioned therethrough. In an example, the base 104 may exhibit the general shape or contours of the skin surface that the base 104 is configured to be coupled with. The base 104 may be flexible, thereby allowing the base 104 to conform to any shape of the skin surface and mitigate the base 104 pulling the on skin surface. The base 104 may extend laterally past the sheath 102 thereby increasing the surface area of the skin of the individual to which the fluid collection assembly 100 may be attached compared to a substantially similar fluid collection assembly 100 that did not include a base.

FIG. 1C is an enlarged cross-sectional schematic of a portion of the base 104, according to an embodiment. As illustrated, the base 104 may include a substrate 134. The substrate 134 includes a top surface 136 and a bottom surface 138. The top surface 136 is closer to the sheath 102 than the bottom surface 138 while the bottom surface 138 is, during use, closer to the skin of the individual than the top surface 136. In an embodiment, a portion of the top surface 136 may be attached to or configured to be attached to the sheath 102.

The substrate 134 may be formed from any suitable material. In an embodiment, the substrate 134 may be formed from fluid impermeable material(s), such as any of the fluid impermeable materials disclosed herein. In such an embodiment, the substrate 134 inhibits bodily fluids from leaking therethrough. In an embodiment, the substrate 134 is formed from a porous material, such as a porous material that is air-permeable and water impermeable (e.g., a hydrophobic porous material). In such an embodiment, the substrate 134 may allow air to flow therethrough thereby encouraging fluid flow through the fluid collection assembly 100 and preventing a suction force provided to the fluid collection assembly 100 from giving the patient a hickie. In an embodiment, the substrate 134 may be formed from a flexible material and/or may exhibit a relatively thin thickness (e.g., less than about 2 mm, less than about 1 mm, less than about 0.5 mm, or in ranges of about 0.25 mm to about 0.75 mm, about 0.5 mm to about 1 mm, about 0.75 mm to about 1.5 mm, or about 1 mm to about 2 mm). The flexibility and/or thickness of the substrate 134 may allow the substrate 134 to be shaped to conform to the shape of the region about the urethral opening (e.g., mons pubis, testicles, etc.) without pulling on the region about the urethral opening.

The base 104 also include a securement body 106. The securement body 106 includes a support 140 that is distinct from the substrate 134. At least a portion of the support 140 is attached to at least a portion of bottom surface 138 of the substrate 134. The support 140 may be attached to the substrate 134 using any suitable technique, such as with an adhesive, ultrasonic welding, etc. Generally, the attachment between the substrate 134 and the support 140 is greater than a maximum attachment between the securement body 106 and the skin of the patient such that detaching the base 104 from the patient is unlikely to detach the support 140 from the substrate 134. It is noted that the securement body 106 and the support 140 may be attached to components of the fluid collection assembly 100 other than or in addition to the base 104, as will be discussed in more detail below.

The support 140 may be formed from any suitable material. In an embodiment, the support 140 may be formed from fluid impermeable material(s), such as any of the fluid impermeable materials disclosed herein. In such an embodiment, the support 140 inhibits bodily fluids from leaking therethrough. In an embodiment, the support 140 is formed from a porous material, such as a porous material that is air-permeable and water impermeable (e.g., a hydrophobic porous material). In such an embodiment, the support 140 may allow air to flow therethrough thereby encouraging fluid flow through the fluid collection assembly 100 and preventing a suction force provided to the fluid collection assembly 100 from giving the patient a hickie. In an embodiment, the support 140 may be formed from a flexible material and/or may exhibit a relatively thin thickness (e.g., less than about 2 mm, less than about 1 mm, less than about 0.5 mm, or in ranges of about 0.25 mm to about 0.75 mm, about 0.5 mm to about 1 mm, about 0.75 mm to about 1.5 mm, or about 1 mm to about 2 mm). The flexibility and/or thickness of the support 140 may allow the support 140 to be shaped to conform to the shape of the region about the urethral opening (e.g., mons pubis, testicles, etc.) without pulling on the region about the urethral opening.

The securement body 106 includes a plurality of fibers 142 extending from support 140. The plurality of fibers 142 are configured to attach the base 104 to the region about the urethral opening. The plurality of fibers 142 exhibit a large surface area due to the relatively small lateral dimensions d (e.g., diameters) thereof, as discussed in more detail below. The large surface area of the fibers 142 cause the fibers 142 to exhibit sufficiently large Van der Waal forces with the region about the urethral opening (e.g., the skin and hair about the urethral opening) that the fibers 142 securely attach the base 104 to the region about the urethral opening. In some embodiments, the fibers 142 may attach the base 104 to the region about the urethral opening as strongly as some chemical adhesives (e.g., hydrogels) when certain forces are applied to the base 104. However, the fibers 142 remain part of the base 104 when the base 104 is detached from the region about the urethral opening unlike chemical adhesives which may leave a film attached to the region about the urethral opening. In an example, since the fibers 142 remain part of the base 104, at least the base 104 may be reused. In such an example, the base 104 may be detached from the region about the urethral opening to allow for examination of the urethral opening and the region about the urethral opening (e.g., regardless if the panels are transparent) and, after examination, the base 104 may be reattached to the region about the urethral opening. Additionally or alternatively, after detaching the base 104 from the region about the urethral opening, the base 104 and, optionally, the sheath 104 may be washed and reused with the same or a new patient. In an example, the fibers 142 may be able to attach the base 104 to the region about the urethral opening when the region about the urethral opening is wet, dry, shaved, or hairy.

The fibers 142 may be selected to exhibit an average lateral dimension d. As used herein, the average lateral dimension d may refer to the average lateral dimension of one fiber 142 at a single location, the average lateral dimension of one fiber 142 averaged along a length thereof, or the average lateral dimension averaged across at least some (e.g., all) of the fibers 142. The average lateral dimension d may be selected to be less than about 10 μm, less than about 8 μm, less than about 6 μm, less than about 5 μm, less than about 4 μm, less than about 3 μm, less than about 2.5 μm, less than about 2 μm, less than about 1.5 μm, less than about 1 μm, less than about 750 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 25 nm, less than about 10 nm, or in ranges of about 5 nm to about 25 nm, about 10 nm to about 50 nm, about 25 nm to about 75 nm, about 50 nm to about 100 nm, about 75 nm to about 150 nm, about 100 nm to about 200 nm, about 150 nm to about 300 nm, about 200 nm to about 400 nm, about 300 nm to about 500 nm, about 400 nm to about 600 nm, about 500 nm to about 750 nm, about 600 nm to about 1 μm, about 750 μm to about 1.5 μm, about 1 μm to about 2 μm, about 1.5 μm to about 3 μm, about 2 μm to about 4 μm, about 3 μm to about 5 μm, about 4 μm to about 7.5 μm, or about 5 μm to about 10 μm. The average lateral dimension d may be selected based on a number of factors.

In an example, the average lateral dimension d may be selected based on the desired adhesion between the securement body 106 and the region about the urethral opening. For instance, decreasing the average lateral dimension d increases the surface area of the fibers 142. The Vander der Waal forces between the fibers 142 and the region about the urethral opening increases as the surface area of the fibers 142 increase and the adhesion between the securement body 106 and the region about the urethral opening increases as the Vander de Waal forces increase. It is noted that the adhesion between the securement body 106 and the region about the urethral opening also depends on other factors, such as the number of fibers per unit of surface area of the support 140 and the length of the fibers 142.

In an example, the average lateral dimension d of the fibers 142 may be selected based on the material that forms the fibers 142. For instance, as previously discussed, the Vander der Waal forces between the fibers 142 and the region about the urethral opening depends on the surface area of the fibers 142 that are proximate to the region about the urethral opening. The surface area of the fibers 142 that are proximate to the region about the urethral opening may depend on the flexibility of the fibers 142. The flexibility of the fibers 142 depends inversely to the average lateral dimension d of the fibers 142 and inversely to the Young's modulus (i.e., modulus of elasticity) of the material that forms the fibers 142. As such, the average lateral dimension d may need to be increased as the Young's modulus of the material that forms the fibers 142 is decreased, and vice versa. In an embodiment, the fibers 142 are formed from a polymer, such as polyimide, polypropylene, polydimethylsiloxane, or any other suitable polymer. In such an embodiment, the average lateral dimension d of the fibers 142 may depend on the molecular weight of the polymer. In an embodiment, the fibers 142 are formed from carbon nanotubes. In such an embodiment, the fibers 142 may exhibit an average lateral dimension d that less than 100 nm, less than 50 nm, or more particularly less than 25 nm.

In an example, the average lateral dimension d of the fibers 142 may depend on the average length l of the fibers 142. For instance, increasing the average length l may increase the likelihood that the fibers 142 become tangled. Tangling the fibers 142 may prevent some of the fibers 142 from pressing against the region of the urethral opening that would press against the region about the urethral opening if not for the tangling. Thus, even though increasing the average length l generally increases the surface area of the fibers 142, increasing the average length l above a threshold value may cause the fibers 142 to become tangled and decrease the adhesion between the securement body 106 and the region about the urethral opening. The threshold value may be increased by increasing the average lateral dimension d of the fibers 142. Thus, increasing the average lateral dimension d allows the lengths of the fibers 142 to be increased without the accompanying issues of entanglement. It is noted that, as used herein, the average length l may refer to the length of a single fiber 142, the length averaged across some of the fibers 142, or the length averaged across all of the fibers 142.

In an embodiment, the average length l of the fibers 142 may be selected to be greater than about 500 nm, greater than about 750 nm, greater than about 1 μm, greater than about 2 μm, greater than about 3 μm, greater than about 5 μm, greater than about 7.5 μm, greater than about 10 μm, greater than about 15 μm, greater than about 25 μm, greater than about 50 μm, greater than about 75 μm, greater than about 100 μm, or in ranges of about 500 nm to about 1 μm, about 750 nm to about 1.5 μm, about 1 μm to about 2 μm, about 1.5 μm to about 3 μm, about 2 μm to about 5 μm, about 3 μm to about 7.5 μm, about 5 μm to about 10 μm, about 7.5 μm to about 15 μm, about 10 μm to about 25 μm, about 15 μm to about 50 μm, about 25 μm to about 75 μm, or about 50 μm to about 100 μm. As previously discussed, the average length l of the fibers 142 may be selected based on the average lateral dimension d and desired surface area of the fibers 142 that is proximate to the region about the urethral opening. The average length l of the fibers 142 may also be selected based on the material(s) that form the fibers 142 since increasing and decreasing the Young's modulus of the material(s) that form the fibers 142 increases and decreases, respectively, the entanglement threshold.

Generally, the average length l of the fibers 142 are greater than the average lateral dimension d. For example, the average length l of the fibers 142 may be selected to be greater than the average lateral dimension d by a factor that is at least about 5, at least about 10, at least about 20, at least about 30, at least about 50, at least about 75, at least about 100, at least about 150, at least about 200, at least about 300, at least about 500, or in ranges of about 5 to 20, about 20 to about 30, about 20 to about 30, about 30 to about 75, about 50 to about 100, about 75 to about 150, about 100 to about 200, about 150 to about 300, or about 200 to about 500.

The fibers 142 may extend from the support 140 at an average angle θ. The average angle θ is an oblique angle. The angle θ is the average smallest angle that may be measured from an outer surface 144 of the support 140 to a portion of the fibers 142 adjacent to the support 140 when no external force is pressing against the fibers 142. The average angle θ may be the angle θ measure between one, some, or all of the fibers 142. The angle θ may be about 1° to about 10°, about 5° to about 15°, about 10° to about 20°, about 15° to about 25°, about 20° to about 30°, about 25° to about 35°, about 30° to about 40°, about 35° to about 45°, about 40° to about 50°, about 45° to about 60°, about 55° to about 75°, or about 70° to about 89°. The average angle θ generally corresponds to the angle between the fibers 142 and the region about the urethral opening assuming the base 104 is sufficiently flexible that the base 104 generally corresponds to the region about the urethral opening. Decreasing the angle θ increases the surface area of the fibers 142 that are proximate to the region about the urethral opening which, in turn, increases the adhesion between the securement body 106 and the region about the urethral opening. Generally, decreasing the average angle θ to be less than about 45° and less than about 30° may allow the fibers 142 to be significantly better at resisting shear forces than if the average angle θ was greater than these values.

The average angle θ may be selected to facilitate attachment, detachment, and securement of the base 104 to the region about the urethral opening. In an example, referring to FIG. 1A, and the base 104 may include a first region 146 and a second region 148. The first region 146 is configured to be attached to the mons pubis while the second region 148 is configured to be attached around the shaft of the penis. The mons pubis exhibits a significantly larger surface area than the area around the shaft of the penis to which the base 104 may be attached. As such, the first region 146 may exhibit a larger surface area than the second region 148. In an embodiment, referring to FIG. 1C, the average angle θ is selected to be less than about 45° (e.g., less than about 30°) and such that at least most of the fibers 142 extend from the support 140 in a direction that generally extends from the second region 148 to the first region 146. In such an embodiment, during use, the base 104 may press into the region about the urethral opening when the patient is lying on the patient's backs such that significant adhesion is not necessary to maintain the base 104 attached to the region about the urethral opening. However, when the patient stands, the average angle θ may resist the shear forces caused by pulling the fluid collection assembly 100 downwards (e.g., towards the feet of the patient). In fact, pulling the fluid collection assembly 100 downwards may decrease the average angle θ thereby increasing adhesion of the base 104 to the region about the urethral opening. However, pulling the fluid collection assembly 100 upwards (e.g., towards the head) or perpendicular to the region about the urethral opening may decrease the average angle θ thereby decreasing adhesion between the securement body 106 and the region about the urethral opening. In other words, the securement body 106 may be more easily detached from the patient (e.g., detached less painfully) when the fluid collection assembly 100 is pulled upward or perpendicular from the region about the urethral opening.

It is noted that the average angle θ does not need to be selected such that the fibers 142 do not extend from the second region 148 to the first region 146. In an example, the average angle θ may be selected such that the fibers 142 extend from the first region 146 to the second region 148. In such an example, a large force pulling downward on the fluid collection assembly 100 may cause the securement body 106 to become detached from the patient which may be beneficial when the downward force is sufficient to cause patient pain if the securement body 106 did not become detached. In an example, the average angle θ may be selected such that the fibers 142 do not extend from the first region 146 to the second region 148 or from the second region 148 to the first region 146 (e.g., the fibers 142 extend in a sideways direction). In such an example, the securement body 106 may be used with a patient that remains mostly in-bed since the downward force applied to the fluid collection assembly 100 caused by standing is not likely to be a factor.

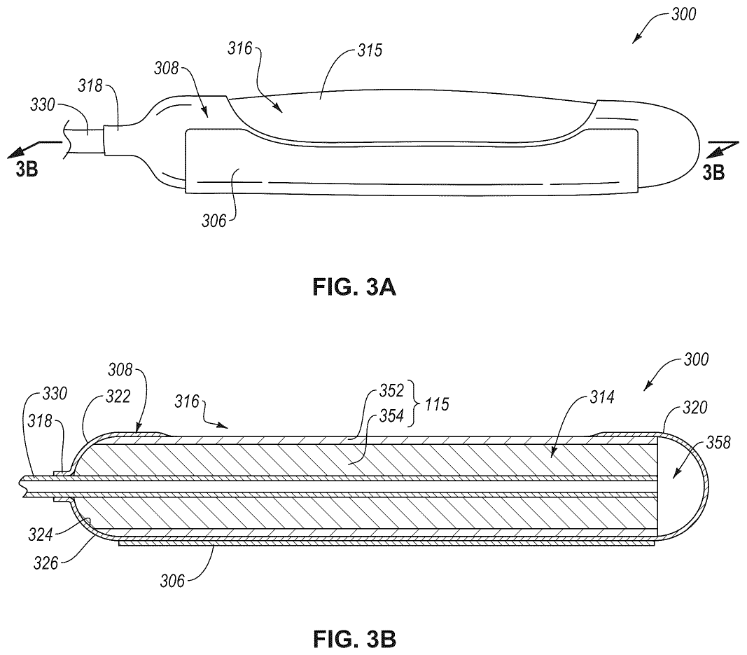

As discussed above, the average angle θ is relative to the outer surface 144 of the support 140. However, as will be discussed in more detail below, the fibers 142 may extend from at least one exterior surface of the fluid collection assembly 100 other than or in addition to the outer surface 144 of the support 140. For example, the fibers 142 may extend from the bottom surface 138 of the substrate 134 (as shown in FIGS. 1E and 1F) or the fluid impermeable barrier 108 (as discussed with regards to FIGS. 3A and 3B). As such, it is noted that the average angle θ is measure relative to the at least one exterior surface of the fluid collection assembly from which the fibers extend.

The securement body 106 may include at least 5,000 fibers per square centimeter of surface area of the outer surface 144 of the support 140 (“f/sc”), at least 10,000 f/sc, at least about 25,000 f/sc, at least about 50,000 f/sc, at least about 75,000 f/sc, at least about 100,000 f/sc, at least about 150,000 f/sc, at least about 200,000 f/sc, at least about 300,000 f/sc, at least about 500,000 f/sc, at least about 750,000 f/sc, at least about 1,000,000 f/sc, at least about 1,500,000 f/sc, at least about 2,500,000 f/sc, at least about 5,000,000 f/sc, at least about 10,000,000 f/sc, at least about 20,000,000 f/sc, or in ranges of about 10,000 f/sc to about 50,000 f/sc, about 25,000 f/sc to about 75,000 f/sc, about 50,000 f/sc to about 100,000 f/sc, about 75,000 f/sc to about 150,000 f/sc, about 100,000 f/sc to about 200,000 f/sc, about 150,000 f/sc to about 300,000 f/sc, about 200,000 f/sc to about 500,000 f/sc, about 300,000 f/sc to about 750,000 f/sc, about 500,000 f/sc to about 1,000,000 f/sc, about 750,000 f/sc to about 1,500,000 f/sc, about 1,000,000 f/sc to about 2,500,000 f/sc, about 1,500,000 f/sc to about 5,000,000 f/sc, about 2,500,000 f/sc to about 10,000,000 f/sc, or about 5,000,000 f/sc to about 20,000,000 f/sc. Increasing and decreasing number of fibers 142 per square centimeter of surface area of the outer surface 144 of the support 140 increases and decreases, respectively, the adhesion between the securement body 106 and the region about the urethral opening. As such, the number of fibers 142 per square centimeter of surface area of the outer surface 144 may be selected based on the desired adhesion, other factors that affect adhesion (e.g., average lateral dimension d, average length l, etc.), and the maximum number of fibers 142 that may reasonably fit within the square centimeter.

In an embodiment, securement body 106 may include one or more branches 150 extending therefrom. The branches 150 may extend from or attached to a portion of the fibers 142 at or near the terminal end thereof. For illustrative purposes (i.e., to more clearly illustrated the fibers 142 and prevent clutter), only one of the fibers 142 are illustrated as having branches 150 extending therefrom. The branches 150 may increase the surface area of the fibers 142 which, in turn, increases the adhesion between the securement body 106 and the region about the urethral opening. The branches 150 exhibit an average diameter and an average length that is significantly less than the average lateral dimension d and the average length l of the fibers 142. For example, the average diameter and/or average length of the branches 150 may be less than the average lateral dimension d and/or average length l of the fibers 142, respectively, by at least about 2, at least about 5, at least about 10, at least about 15, at least about 20, at least about 30, at least about 40, at least about 50, or in ranges of about 2 to about 10, about 5 to about 15, about 10 to about 20, about 15 to about 30, about 20 to about 40, or about 30 to about 50. In an example, the average diameter of the branches 150 may exhibit be less than about less than about 2 μm, less than about 1.5 μm, less than about 1 μm, less than about 750 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 25 nm, less than about 10 nm, less than about 5 nm or in ranges of about 1 nm to about 10 nm, about 5 nm to about 25 nm, about 10 nm to about 50 nm, about 25 nm to about 75 nm, about 50 nm to about 100 nm, about 75 nm to about 150 nm, about 100 nm to about 200 nm, about 150 nm to about 300 nm, about 200 nm to about 400 nm, about 300 nm to about 500 nm, about 400 nm to about 600 nm, about 500 nm to about 750 nm, about 600 nm to about 1 μm, about 750 μm to about 1.5 μm, or about 1 μm to about 2 μm. In an example, the length of the branches 150 may be less than about 500 nm, less than about 750 nm, less than about 1 μm, less than about 2 μm, less than about 3 μm, less than about 5 μm, less than about 7.5 μm, less than about 10 μm, less than about 15 μm, less than about 25 μm, less than about 50 μm, less than about 75 μm, less than about 100 μm, or in ranges of about 500 nm to about 1 μm, about 750 nm to about 1.5 μm, about 1 μm to about 2 μm, about 1.5 μm to about 3 μm, about 2 μm to about 5 μm, about 3 μm to about 7.5 μm, about 5 μm to about 10 μm, about 7.5 μm to about 15 μm, about 10 μm to about 25 μm, or about 15 μm to about 50 μm. In an embodiment, one or more of the branches 150 may branch into a plurality of additional branches.

Referring to FIG. 1C, the fibers 142 are distinct from the support 140. As such, the fibers 142 may be formed on the support 140 or formed separately from the support 140 and attached to the support 140 after forming the fibers 142. In an example, the fibers 142 may be formed via an electro-spinning technique and attached to the support 140. In an example, the fibers 142 may be grown on the support 140, for instance, using a chemical vapor deposition (“CVD”) or physical vapor deposition (“PVD”) technique. In such an example, the support 140 may be masked prior to growing the fibers 142 on the support 140. The mask may be removed from the support 140 after growing the fibers 142 or the mask may remain. In an example, the fibers 142 may be formed using an overmolding and/or micromolding technique.

The base 104 may exhibit a different structure than the structure illustrated in FIG. 1C depending on the technique used to form the fibers 142. FIGS. 1D to 1F are cross-sectional schematics of different bases that may be used with the fluid collection assembly 100, according to different embodiments. Referring to FIG. 1D, the base 104d includes a substrate 134d that is the same or substantially similar to the substrate 134 of FIG. 1C. The base 104d also include a securement body 106d. The securement body 106d includes a support 140d and a plurality of fibers 142d. The support 140d and the fibers 142d may be the same or substantially similar to the support 140 and the fibers 142 of FIG. 1C, respectively, (e.g., formed from any of the support and/or fibers materials disclosed herein) except that the support 140d and the fibers 142d are integrally formed with each other. An example of forming the securement body 106d includes providing a piece of material. Portions of the material may be selectively removed therefrom to form the support 140d and the fibers 142d. The portion of the material may be selectively removed using a photolithography technique or any other suitable technique. It is noted that a mask may be formed on the portions of the material that form the fibers 142d to prevent the removal of such portions of the material. The masks may be removed after forming the fibers 142d.

Referring to FIG. 1E, the base 104e may include a substrate 134e and a securement body 106e attached to the substrate 134e. The securement body 106e include one or more fibers 142e that extend directly from and are distinct from the substrate 134e. In other words, at least a portion of the securement body 106e does not include a support. The fibers 142e may be formed directly on the substrate 134e in any of the same manners that the fibers 142 are formed on the support 140 of FIG. 1C. For example, preformed fibers (e.g., electro-spun fibers) may be directly attached to the substrate 134e or the fibers 142e may be grown on the substrate 134c (e.g., using CVD or PVD).

Referring to FIG. 1F, the base 104f may include a substrate 134f and a securement body 106f that are integrally formed together. For example, the securement body 106f may include a plurality of fibers 142f that are integrally formed with the substrate 134f. The base 104f may be formed by providing a piece of material and selectively removing portions of the material to form the fibers 142f. In an example, portions of the material may be removed via photolithography. In such an example, the material may be masked to prevent removal of portions of the material that form the fibers 142f. The mask may be removed after forming the fibers 142f.

Referring back to FIGS. 1A and 1B, it is noted that the securement bodies disclosed herein may be formed on at least a portion of the fluid impermeable barrier 108 instead of or in addition to the base 104. For example, the securement body formed on at least a portion of the fluid impermeable barrier 108 may include fibers attached to a support that is attached to the fluid impermeable barrier 108, integrally formed with the support that is attached to the fluid impermeable barrier 108, directly attached to the fluid impermeable barrier 108, or integrally formed with the fluid impermeable barrier 108.

As previously discussed, the fluid collection assembly 100 includes a conduit 130. The conduit 130 may be the same or substantially similar to any of the assembly tubes disclosed herein. An inlet of the conduit 130 may be located at or near the second end region 122 of the sheath 102 which is expected to be the gravimetrically low point of the chamber 114 when worn by a user. Locating the inlet of the conduit 130 at or near the second end region 122 of the sheath 102 enables the conduit 130 to receive more of the bodily fluids than if the inlet of the conduit 130 was located elsewhere and reduce the likelihood of pooling (e.g., pooling of the bodily fluids may cause microbe growth and foul odors). For instance, the bodily fluids in porous material 115 flow into the porous material 115 due to capillary forces. However, the bodily fluids may exhibit a preference to flow in the direction of gravity, especially when at least a portion of the porous material 115 is saturated with the bodily fluids. Accordingly, the inlet of the conduit 130 may be located in the fluid collection assembly 100 in a position expected to be the gravimetrically low point in the fluid collection assembly 100 when worn by a user.

In an example, the conduit 130 is configured to be at least insertable into the chamber 114, such as into the penis receiving area 128. In such an example, the conduit 130 may include one or more markers (not shown) on an exterior thereof that are located to facilitate insertion of the conduit 130 into the chamber 114. For example, the conduit 130 may include one or more markings thereon that are configured to prevent over or under insertion of the conduit 130. In another example, the conduit 130 may include one or more markings thereon that are configured to facilitate correct rotation of the conduit 130 relative to the chamber 114. The one or more markings may include a line, a dot, a sticker, or any other suitable marking.

The conduit 130 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 130 may include silicon or latex. In some examples, the conduit 130 may include one or more portions that are resilient, such as to by having one or more of a diameter or wall thickness that allows the conduit 130 to be flexible.

As described in more detail below, the conduit 130 is configured to be coupled to, and at least partially extend between, one or more of the fluid storage container (not shown) and the vacuum source (not shown). In some examples, the vacuum source may be remotely located from the fluid collection assembly 100. In such examples, the conduit 130 may be fluidly connected to the fluid storage container, which may be disposed between the vacuum source and the fluid collection assembly 100.

During operation, a male using the fluid collection assembly 100 may discharge bodily fluids (e.g., urine) into the chamber 114. The bodily fluids may pool or otherwise be collected in the chamber 114. At least some of the bodily fluids may be pulled through the interior of the conduit 130 via the inlet. The fluid may be drawn out of the fluid collection assembly 100 via the vacuum/suction provided by the vacuum source. Further examples of male fluid collection assemblies are disclosed in U.S. Provisional Patent Application No. 63/067,542 filed on Aug. 19, 2020, the disclosure of which is incorporated herein, in its entirety, by this reference.

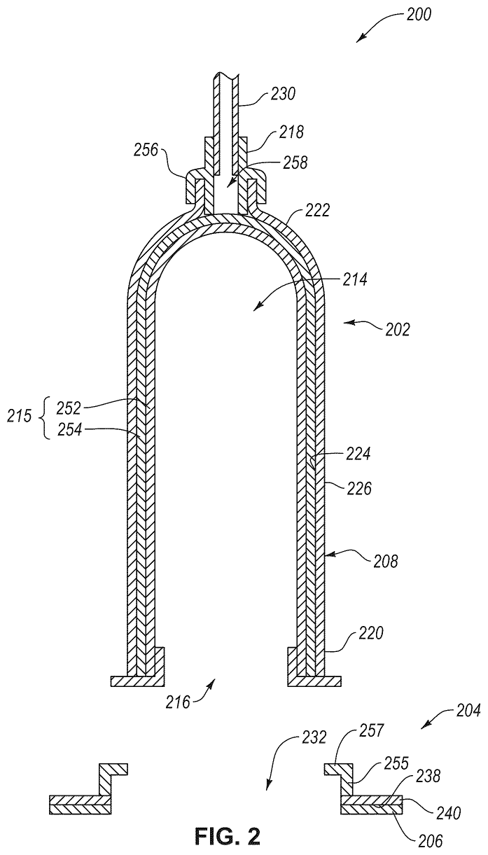

The securement body disclosed herein may be used with other male fluid collection assemblies. For example, FIG. 2 is a cross-sectional schematic of a fluid collection assembly 200, according to an embodiment. The fluid collection assembly 200 is a male fluid collection assembly configured to receive one or more bodily fluids from a male urethral opening. Except as otherwise disclosed herein, the fluid collection assembly 200 is the same or substantially similar to any of the fluid collection assemblies disclosed herein.

The fluid collection assembly 200 includes a sheath 202 and a base 204 and a sheath 202. The sheath 202 includes (e.g., may be formed from) a fluid impermeable barrier 208 that is sized and shaped to fit into the hollowed region of the base 204. For example, the sheath 202 may be generally tubular or cup-shaped, as shown. The generally tubular or cup-shaped fluid impermeable barrier 208 may at least partially define the outer surface 226 of the sheath 202. The fluid impermeable barrier 208 may be similar or identical to the fluid impermeable barrier 108 as disclosed herein, in one or more aspects. For example, the fluid impermeable barrier 208 may be constructed of any of the materials disclosed herein for the fluid impermeable barrier 108. The fluid impermeable barrier 208 at least partially defines the chamber 214. For example, the inner surface 224 of the fluid impermeable barrier 208 at least partially defines the perimeter of the chamber 214. The chamber 214 may be similar or identical to the chamber 114 in one or more aspects. For example, the chamber 214 may at least temporarily retain fluids therein. As shown, the fluid collection assembly 200 may include the porous material 215 therein. The porous material 215 may be similar or identical to the porous material 115 in one or more aspects. In an example, the porous material 215 may include one or more of a fluid permeable membrane 252 or a fluid permeable support 254. In an example, the porous material 215 may include any of the other porous material disclosed herein. The fluid impermeable barrier 208 may also define an opening 216 extending through the fluid impermeable barrier 208 that is configured to have a male urethra positioned therethrough.

The sheath 202 also includes at least a portion of the conduit 230 therein, such as at least partially disposed in the chamber 214. For example, the conduit 230 may extend from the sheath 202 at the second end region 222 at least partially towards a first end region 220 at least proximate to the aperture 232. The first end region 220 may be disposed near or on the skin around the male urethra (e.g., on the penis or pubic area therearound).

In some examples, the fluid impermeable barrier 208 may be constructed of a material and/or have a thickness that allows the sheath 202 to collapse when placed under vacuum, such as to remove air around a penis in the fluid collection assembly 200 during use. In such examples, the conduit 230 may extend only to or into the second end region 222 in the chamber 214 (e.g., not through to the area adjacent the opening 216). In such examples, urine may be collected and removed from the fluid collection assembly 200 at the first end region 220. It is noted that the porous material 215 may not collapse when the sheath 202 collapses thereby allowing bodily fluids to flow through the fluid collection assembly 200.

In an example, portions of the chamber 214 may be substantially empty due to the varying sizes and rigidity of the male penis. However, in some examples, the outermost regions of the chamber 214 (e.g., periphery of the interior regions of the sheath 202) may include porous material 215 (e.g., one or more of the fluid permeable membrane 252 and fluid permeable support 254). For example, the porous material 215 may be bonded to the inner surface 224 of the fluid impermeable barrier 208. The porous material 215 may be positioned (e.g., at the distal end of the chamber 214) to blunt a stream of urine from the male urethra thereby limiting splashing and/or to direct the bodily fluids to a selected region of the chamber 214. Since the chamber 214 is substantially empty (e.g., substantially all of the chamber 214 forms a reservoir), the fluids are likely to pool at a gravimetrically low point of the chamber 214. The gravimetrically low point of the chamber 214 may be at an intersection of the skin of an patient and the fluid collection assembly 200, a corner formed in the sheath 202, or another suitable location depending on the orientation of the patient.

The porous material 215 may include any of the porous material(s) disclosed herein, In an example, as previously discussed, the porous material 215 may include one or more of the fluid permeable membrane 252 or the fluid permeable support 254. One or more of the fluid permeable membrane 252 or the fluid permeable support 254 may be disposed between the fluid impermeable barrier 208 and a penis inserted into the chamber 214. The fluid permeable membrane 252 may be positioned between the fluid impermeable barrier 208 and a penis inserted into the chamber 214, such as between the fluid permeable support 254 and penis of a patient as shown. The fluid permeable support 254 may be positioned between the fluid permeable membrane 252 and the fluid impermeable barrier 208. The inner surface 224, optionally including the end of the chamber 214 substantially opposite the opening 216, may be covered with one or both the fluid permeable membrane 252 or the fluid permeable support 254. The fluid permeable support 254 or the fluid permeable membrane 252 may be affixed (e.g., adhered) to the fluid impermeable barrier 208. The fluid permeable support 254 or the fluid permeable membrane 252 may be affixed to each other. In some examples, the porous material 215 only includes the fluid permeable membrane 252 or the fluid permeable support 254.

The base 204 is sized, shaped, and made of a material to be coupled to skin that surrounds the male urethra and have the male urethra positioned therethrough. For example, the base 204 may include an substrate 234 that defines an aperture 232 in the base 204. The substrate 234 is sized and shaped to be positioned around the male urethra (e.g., positioned around and/or over the penis) and the aperture 232 may be configured to have the male urethra positioned therethrough. The substrate 234 may also be sized, shaped, made of a material, or otherwise configured to be coupled (e.g., adhesively attached, such as with a hydrogel adhesive) to the skin around the male urethra (e.g., around the penis). In an example, the substrate 234 may exhibit the general shape or contours of the skin surface that the substrate 234 is selected to be coupled with. The substrate 234 may be flexible thereby allowing the substrate 234 to conform to any shape of the skin surface. The base 204 may include a longitudinally extending flange 255 extending from the substrate 234 and a laterally extending flange 257 extending inwardly from the longitudinal extending flange 255. The longitudinally extending flange 255 and the laterally extending flange 257 define a hollowed region that is configured to receive (e.g., seal against) the sheath 202.

The base 204 also includes at least one securement body 206. For example, the securement body 206 may be attached to at least a portion of a bottom surface 238 of the substrate 234. The securement body 206 may be the same or substantially similar to any of the securement body disclosed herein. For example, the securement body 206 includes a plurality of fibers (not shown) that are configured to be attach the base 204 to a region about the urethral opening. The fibers may be attached to a support that is attached to the bottom surface 238 of the substrate 234 (as shown in FIG. 1C), integrally formed with the support (as shown in FIG. 1D), directly attached to the substrate 234 (as shown in FIG. 1E), or integrally formed with the substrate 234 (as shown in FIG. 1F).

In some examples, the fluid collection assembly 200 includes a cap 256 at a second end region 222. The cap 256 defines an interior channel through which the fluids may be removed from the fluid collection assembly 200. The interior channel is in fluid communication with the chamber 214. The cap 256 may be disposed over at least a portion of the second end region 222 of one or more of the fluid impermeable barrier 208 or the porous material 215. The cap 256 may be made of a polymer, rubber, or any other fluid impermeable material. The cap 256 may be attached to one or more of the fluid impermeable barrier 208, the porous material 215, or the conduit 130. The cap 256 may cover at least a portion of the second end region 222 of the fluid collection assembly 200. The cap 256 may laterally extend a distance from the sheath 202. The cap 256 defines a fluid outlet 218 that is sized and configured to receive and fluidly seal against the conduit 230, such as within the interior channel. The conduit 230 may extend a distance within or through the cap 256, such as to the porous material 215, through the porous material 215, or to a point set-off from the porous material 215. In the latter example, the interior channel of the cap 256 may define a reservoir 258 therein. In some examples (not shown), the cap 256 may be omitted.

The reservoir 258 is an unoccupied portion of fluid collection assembly 200 such as in the cap 256 and is void of other material. In some examples, the reservoir 258 is defined at least partially by the porous material 215 and the cap 256. During use, the fluids that are in the chamber 214 may flow through the porous material 215 to the reservoir 258. The reservoir 258 may store at least some of the fluids therein and/or position the fluids for removal by the conduit 230. In some examples, at least a portion of the porous material 215 may extend continuously between at least a portion of the opening of the interior channel and chamber 214 to wick any fluid from the opening directly to the reservoir 258.

The base 204, the sheath 202, the cap 256, and the conduit 230 may be attached together using any suitable method. For example, at least two of the base 204, the sheath 202, the cap 256, or the conduit 230 may be attached together using at least one of an interference fit, an adhesive, stitching, welding (e.g., ultrasonic welding), tape, any other suitable method, or combinations thereof.

In some examples (not shown), the fluid collection assembly 200 may have a one piece design, with one or more of the sheath 202, the base 204, and the cap 256 being a single, integrally formed piece.

Further examples of male fluid collection assemblies that may be used here are disclosed in U.S. patent application Ser. No. 16/433,773 filed on Jun. 6, 2019, the disclosure of which is incorporated herein, in its entirety, by this reference.

The securement body disclosed herein may be used with a female fluid collection assembly. FIG. 3A is an isometric view of a fluid collection assembly 300, according to an embodiment. FIG. 3B is a cross-sectional schematic of the fluid collection assembly 300 taken along plane 3B-3B shown in FIG. 3A, according to an embodiment. The fluid collection assembly 300 is a female fluid collection assembly that is configured to be disposed adjacent to a female urethral opening. Except as otherwise disclosed herein, the fluid collection assembly 300 is the same or substantially similar to any of the fluid collection assemblies disclosed herein. The fluid collection assembly 300 includes a fluid impermeable barrier 308, at least one porous material 315 disposed in a chamber 314 defined by the fluid impermeable barrier 308, at least one securement body 306, and an optional conduit 330 at least partially disposed within the chamber 314.

The fluid impermeable barrier 308 at least partially defines a chamber 314 (e.g., interior region) and an opening 316. For example, the interior surface(s) 324 of the fluid impermeable barrier 308 at least partially defines the chamber 314 within the fluid collection assembly 300. The fluid impermeable barrier 308 temporarily stores the bodily fluids in the chamber 314. The fluid impermeable barrier 308 may be formed of any suitable fluid impermeable material(s), such as any of the fluid impermeable materials disclosed herein. As such, the fluid impermeable barrier 308 substantially prevents the bodily fluids from passing through the fluid impermeable barrier 308. In an example, the fluid impermeable barrier 308 may be air permeable and fluid impermeable. In such an example, the fluid impermeable barrier 308 may be formed of a hydrophobic material that defines a plurality of pores. At least one or more portions of at least an outer surface 326 of the fluid impermeable barrier 308 may be formed from a soft and/or smooth material, thereby reducing chaffing.

In some examples, the fluid impermeable barrier 308 may be tubular (ignoring the opening 316), such as substantially cylindrical (as shown), oblong, prismatic, or flattened tubes. During use, the outer surface 326 of the fluid impermeable barrier 308 may contact the patient. The fluid impermeable barrier 308 may be sized and shaped to fit in the gluteal cleft between the legs of a female user.

The opening 316 provides an ingress route for fluids to enter the chamber 314. The opening 316 may be defined by the fluid impermeable barrier 308 such as by an inner edge of the fluid impermeable barrier 308. For example, the opening 316 is formed in and extends through the fluid impermeable barrier 308, from the outer surface 326 to the inner surface 324, thereby enabling bodily fluids to enter the chamber 314 from outside of the fluid collection assembly 300. The opening 316 may be an elongated hole in the fluid impermeable barrier 308. For example, the opening 316 may be defined as a cut-out in the fluid impermeable barrier 308. The opening 316 may be located and shaped to be positioned adjacent to a female urethra.

The fluid collection assembly 300 may be positioned proximate to the female urethral opening and the bodily fluids may enter the chamber 314 of the fluid collection assembly 300 via the opening 316. The fluid collection assembly 300 is configured to receive the bodily fluids into the chamber 314 via the opening 316. When in use, the opening 316 may have an elongated shape that extends from a first location below the urethral opening (e.g., at or near the anus or the vaginal opening) to a second location above the urethral opening (e.g., at or near the top of the vaginal opening or the pubic hair).

The opening 316 may have an elongated shape because the space between the legs of a female is relatively small when the legs of the female are closed, thereby only permitting the flow of the bodily fluids along a path that corresponds to the elongated shape of the opening 316 (e.g., longitudinally extending opening). The opening 316 in the fluid impermeable barrier 308 may exhibit a length that is measured along the longitudinal axis of the fluid collection assembly 300 that may be at least about 20% of the length of the fluid collection assembly 300, such as about 25% to about 50%, about 40% to about 60%, about 50% to about 75%, about 65% to about 85%, or about 75% to about 95% of the length of the fluid collection assembly 300.

The opening 316 in the fluid impermeable barrier 308 may exhibit a width that is measured transverse to the longitudinal axis of the fluid collection assembly 300 that may be at least about 30% of the circumference of the fluid collection assembly 300, such as about 25% to about 50%, about 40% to about 60%, about 50% to about 75%, about 65% to about 85%, or about 75% to about 300% of the circumference of the fluid collection assembly 300. The opening 316 may exhibit a width that is greater than 50% of the circumference of the fluid collection assembly 300 since the vacuum (e.g., suction) through the conduit 330 pulls the fluid through the porous material 315 and into the conduit 330. In some examples, the opening 316 may be vertically oriented (e.g., having a major axis parallel to the longitudinal axis of the fluid collection assembly 300). In some examples (not shown), the opening 316 may be horizontally oriented (e.g., having a major axis perpendicular to the longitudinal axis of the fluid collection assembly 300). In an example, the fluid impermeable barrier 308 may be configured to be attached to the patient, such as adhesively attached (e.g., with a hydrogel adhesive) to the patient. According to an example, a suitable adhesive is a hydrogel layer.

In some examples, the fluid impermeable barrier 308 may define an fluid outlet 318 sized to receive the conduit 330. The at least one conduit 330 may be disposed in the chamber 314 via the fluid outlet 318. The fluid outlet 318 may be sized and shaped to form an at least substantially fluid tight seal against the conduit 330 or the at least one tube thereby substantially preventing the bodily fluids from escaping the chamber 314.

The fluid impermeable barrier 308 may include markings thereon, such as one or more markings to aid a user in aligning the fluid collection assembly 300 on the patient. For example, a line on the fluid impermeable barrier 308 (e.g., opposite the opening 316) may allow a healthcare professional to align the opening 316 over the urethra of the patient. In examples, the markings may include one or more of alignment guide or an orientation indicator, such as a stripe or hashes. Such markings may be positioned to align the fluid collection assembly 300 to one or more anatomical features such as a pubic bone, etc.

The fluid collection assembly 300 includes porous material 315 disposed in the chamber 314. The porous material 315 may cover at least a portion (e.g., all) of the opening 316. The porous material 315 is exposed to the environment outside of the chamber 314 through the opening 316. In an embodiment, the porous material 315 may be configured to wick any bodily fluids away from the opening 316, thereby preventing the bodily fluids from escaping the chamber 314. In an embodiment, the porous material 315 may include at least one absorbent or adsorbent material.

The porous material 315 may include any of the porous material disclosed herein. For example, the porous material 315 may include a fluid permeable membrane 352 and a fluid permeable support 354 disposed in the chamber 314. The fluid permeable membrane 352 may cover at least a portion (e.g., all) of the opening 316. The fluid permeable membrane 352 may be composed to wick the bodily fluids away from the opening 316, thereby preventing the bodily fluids from escaping the chamber 314. The fluid permeable membrane 352 may include any of the fluid permeable membrane materials disclosed herein.

The fluid permeable support 354 is configured to support the fluid permeable membrane 352 since the fluid permeable membrane 352 may be formed from a relatively foldable, flimsy, or otherwise easily deformable material. For example, the fluid permeable support 354 may be positioned such that the fluid permeable membrane 352 is disposed between the fluid permeable support 354 and the fluid impermeable barrier 308. As such, the fluid permeable support 354 may support and maintain the position of the fluid permeable membrane 352. The fluid permeable support 354 may include any material that may wick the bodily fluids, such as any of the fluid permeable membrane materials or fluid permeable support materials disclosed herein. For example, the fluid permeable membrane material(s) may be utilized in a more dense or rigid form than in the fluid permeable membrane 352 when used as the fluid permeable support 354. The fluid permeable support 354 may be formed from any fluid permeable material that is less deformable than the fluid permeable membrane 352.

In some examples, the fluid permeable membrane 352 may be optional. For example, the porous material 315 may include only the fluid permeable support 354. In some examples, the fluid permeable support 354 may be optionally omitted from the fluid collection assembly 300. For example, the porous material 315 may only include the fluid permeable membrane 352.

The fluid permeable support 354 may have a greater ability to wick the bodily fluids than the fluid permeable membrane 352, such as to move the bodily fluids inwardly from the outer surface of the fluid collection assembly 300. In some examples, the porous ability of the fluid permeable support 354 and the fluid permeable membrane 352 may be substantially the same.

The fluid permeable membrane 352 and the fluid permeable support 354 may at least substantially completely fill the portions of the chamber 314 that are not occupied by the conduit 330. In some examples, the fluid permeable membrane 352 and the fluid permeable support 354 may not substantially completely fill the portions of the chamber 314 that are not occupied by the conduit 330. In such an example, the fluid collection assembly 300 includes the reservoir 358 disposed in the chamber 314.

The reservoir 358 is a substantially unoccupied portion of the chamber 314. The reservoir 358 may be defined between the fluid impermeable barrier 308 and one or both of the fluid permeable membrane 352 and fluid permeable support 354. The bodily fluids that are in the chamber 314 may flow through the porous material 315 to the reservoir 358. The fluid impermeable barrier 308 may retain the bodily fluids in the reservoir 358. While depicted in the first end region 320, the reservoir 358 may be located in any portion of the chamber 314 such as the second end region 322. The reservoir 358 may be located in a portion of the chamber 314 that is designed to be located in a gravimetrically low point of the fluid collection assembly when the fluid collection assembly is worn.

In some examples (not shown), the fluid collection assembly 300 may include multiple reservoirs, such as a first reservoir that is located at the portion of the chamber 314 closest to the inlet of the conduit 330 (e.g., first end region 320) and a second reservoir that is located at the portion of the of the chamber 314 that is at or near second end region 322). In another example, the fluid permeable support 354 is spaced from at least a portion of the conduit 330, and the reservoir 358 may be the space between the fluid permeable support 354 and the conduit 330.

The conduit 330 may be at least partially disposed in the chamber 314. The conduit 330 may be used to remove the bodily fluids from the chamber 314. The conduit 330 (e.g., a tube) includes an inlet of the conduit 330 and an outlet 312 positioned downstream from the inlet of the conduit 330. The outlet 312 may be operably coupled to a suction source, such as a vacuum pump for withdrawing fluid from the chamber 314 through the conduit 330. For example, the conduit 330 may extend into the fluid impermeable barrier 308 from the second end region 322 and may extend to the first end region 320 to a point proximate to the reservoir 358 therein such that the inlet of the conduit 330 is in fluid communication with the reservoir 358. The conduit 330 fluidly couples the chamber 314 with the fluid storage container (not shown) or the vacuum source (not shown).

The conduit 330 may extend through a bore in the fluid permeable membrane 352 and/or fluid permeable support 354, such as into the reservoir 358. For example, the inlet of the conduit 330 may be extend into or be positioned in the reservoir 358. In the illustrated embodiment, the conduit 330 is at least partially disposed in the reservoir 358. In some examples (not shown), the conduit 330 may enter the chamber 314 in the distal end region and the inlet of the conduit 330 of the conduit 330 may be disposed in the distal end region (e.g., in the reservoir 358). The bodily fluids collected in the fluid collection assembly 300 may be removed from the chamber 314 via the conduit 330.

In some examples, the inlet of the conduit 330 may not extend into the reservoir 358. In such examples, the inlet of the conduit 330 may be disposed within the porous material 315 (fluid permeable membrane 352 and/or fluid permeable support 354) or at a terminal end thereof. For example, an end of the conduit 330 may be coextensive with or recessed within the fluid permeable membrane 352 and/or fluid permeable support 354.

During use, the first end region 320 may be the gravimetrically low point of the chamber 314. As such, locating the inlet of the conduit 330 at or near a location expected to be the gravimetrically low point of the chamber 314 when worn by a patient enables the conduit 330 to receive more of the bodily fluids than if inlet of the conduit 330 was located elsewhere and reduce the likelihood of pooling (e.g., pooling of the bodily fluids may cause microbe growth and foul odors). For instance, as previously discussed, the bodily fluids in the fluid permeable membrane 352 and the fluid permeable support 354 may flow in any direction due to capillary forces. However, the bodily fluids may exhibit a preference to flow in the direction of gravity, especially when at least a portion of the fluid permeable membrane 352 and/or the fluid permeable support 354 is saturated with the bodily fluids. Accordingly, one or more of the inlet of the conduit 330 or the reservoir 358 may be located in the fluid collection assembly 300 in a position expected to be the gravimetrically low point in the fluid collection assembly 300 when worn by a patient, such as the first end region 320.

As previously discussed, the fluid collection assembly 300 may include at least one securement body 306. The securement body 306 may be configured to be attached to the skin of the patient, such as at least one of the thighs or the region about the urethral opening. As such, the securement body 206 may be attached to or formed on at least a portion of an outer surface 326 of the fluid impermeable barrier 308 that, during use, may contact the skin of the patient. The securement body 306 may be the same or substantially similar to any of the securement body disclosed herein. As such, the securement body 306 includes a plurality of fibers that are configured to be attach the securement body to the skin of a patient.

In an embodiment, the securement body 306 is distinct from the fluid impermeable barrier 308. In such an embodiment, the securement body 306 may include at least one of a plurality of fibers attached to or integrally formed with a support, similar to what is shown in FIGS. 1C and 1D, respectively. In such an embodiment, the support may be attached to the fluid impermeable barrier 308 using any suitable technique, such as with a chemical adhesive, a dry adhesive, etc. in an embodiment, the securement body 306 may include a plurality of fibers at least one of attached directly to the fluid impermeable barrier 308 or integrally formed with the fluid impermeable barrier 308, similar to what is shown in FIGS. 1E and 1F, respectively.

Other embodiments of fluid impermeable barriers, fluid permeable membranes, fluid permeable supports, chambers, and their shapes and configurations are disclosed in U.S. Pat. No. 10,973,678 filed on Jun. 2, 2017; U.S. Pat. No. 10,390,989 filed on Sep. 8, 2016; and U.S. Pat. No. 10,226,376 filed on Jun. 3, 2017, the disclosure of each of which is incorporated herein, in its entirety, by this reference.

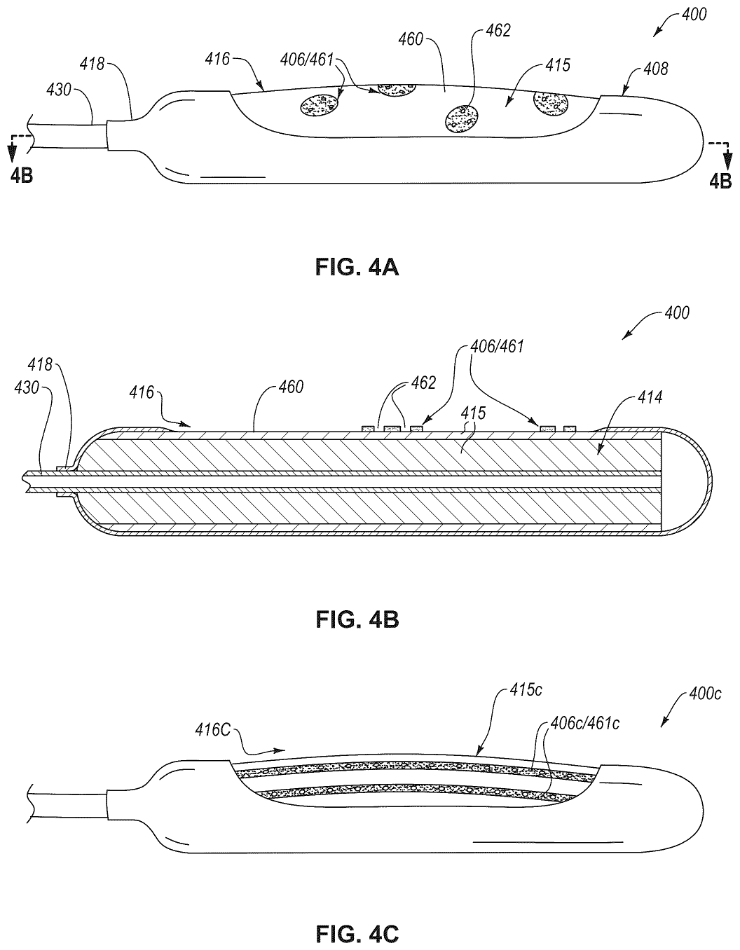

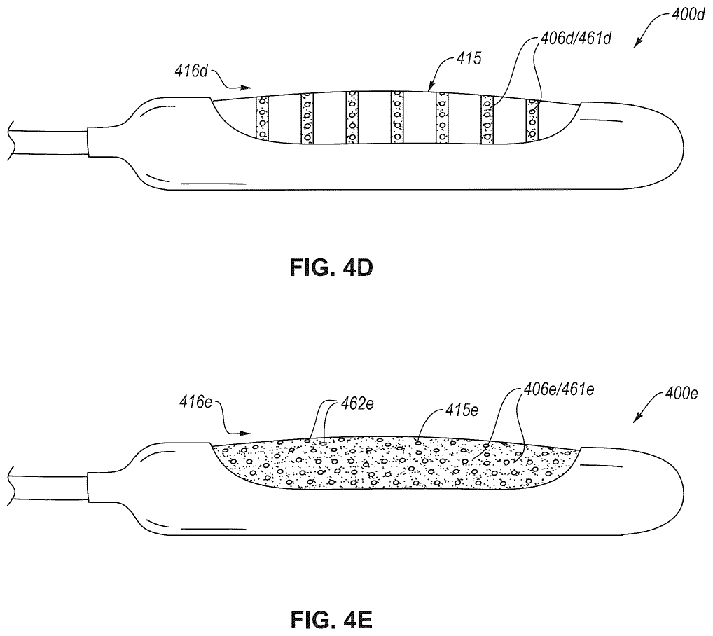

The securement body illustrated in FIGS. 1A-3B include a plurality of fibers. However, the securement body may include one or more additional types of securement bodies, such at least one friction material, instead of or in addition to the securement bodies illustrated in FIGS. 1A-3B. For example, FIG. 4A is an isometric view of a fluid collection assembly 400 that includes at least one friction material 461, according to an embodiment. FIG. 4B is a cross-sectional schematic of the fluid collection assembly 400 taken along plane 4B-4B shown in FIG. 4A, according to an embodiment. The fluid collection assembly 400 is an example of a female fluid collection assembly. As such, except as otherwise disclosed herein, the fluid collection assembly 400 may be the same or substantially similar to the fluid collection assembly 300 shown in FIGS. 3A and 3B. For example, the fluid collection assembly 400 may include a fluid impermeable barrier 408 that defines a chamber 414, at least one opening 416, and a fluid outlet 418. The fluid collection assembly 400 may include at least one porous material 415 disposed in the chamber 414 and an optional conduit 430. Except as otherwise disclosed herein, the fluid impermeable barrier 408, the porous material 415, and the conduit 430 may be the same or substantially similar to any of the fluid impermeable barriers, porous materials, and conduits, respectively, disclosed herein.

The porous material 415 includes a contact surface 460 that is configured to contact the patient during use. The contact surface 460 may include the portion of the porous material 415 that extends across the opening 416.

The fluid collection 400 includes at least one securement body 406. The securement body 406 includes at least one friction material 461 disposed on at least a portion of the contact surface 460. The contact surface 460 exhibits a first coefficient of friction and the friction material 461 exhibits a second coefficient of friction that is greater than the first coefficient of friction. As such, the friction material 461 better prevents movement of the fluid collection assembly 400 when the opening is adjacent to the region about the urethral opening of the patient than if the fluid collection assembly 400 did not include the friction material 461. In is noted that, as used herein, the coefficient of friction refers to the coefficient of static friction of the material (e.g., the contact surface 460 or the friction material 461) against the against the skin of the patient when the skin is at least one of dry or moist.

The friction material 461 includes a material disposed on the contact surface 460 of the porous material 415. The material of the friction material 461 is different than the material that forms the porous material 415 which, at least in part, causes the friction material 461 to exhibit a coefficient of friction that is greater than the contact surface 460. In an example, the friction material 461 includes at least one elastomer, such as at least one of silicone, nitrile, rubber, neoprene, or another elastomer.

The friction material 461 may be disposed on the contact surface 460 using any suitable technique. In an example, the friction material 461 may include an adhesive on the surface of the friction material 461 that contacts the contact surface 460. In an example, the surface of the friction material 461 that contacts the contact surface 460 may be partially melted and the friction material 461 may be pressed into the contact surface 460. At least some of the melted portions of the friction material 461 may flow into and at least partially occupy some of the pores of the porous material 415 thereby attaching the friction material 461 to the porous material 415. In an example, the friction material 461 may be applied to the contact surface 460 while in a melted (e.g., liquid) state. Some of the melted friction material 461 may flow into and at least partially occupy some of the pores of the porous material 415 thereby attaching the friction material 461 to the porous material 415.

As previously discussed, the friction material 461 is disposed on the contact surface 460 of the porous material 415. As such, the friction material 461 may block portions of the porous material 415 that extend across the opening 416. In some embodiments, blocking portions of the porous material 415 that extend across the opening 416 may at least one of limit the amount of bodily fluids that may enter the chamber (not shown) or cause the bodily fluids to splash, either or which may cause bodily fluids to leak or otherwise inhibit the functionality of the fluid collection assembly 400. However, the friction material's 461 the ability to better maintain the position of the fluid collection assembly 400 against the region about the urethral opening still decreases the likelihood that the bodily fluids leak even though the friction material 461 may obstruct portions of the porous material 415 and cause splashing.

In an embodiment, the friction material 461 may define one or more passageways 462 (e.g., pores, voids, etc.) extending therethrough. The passageways 462 allow the bodily fluids to flow through the friction material 461 to the contact surface 460 of the porous material 415 and decrease splashing of the bodily fluids. Thus, the passageways 462 may further prevent bodily fluids from leaking from the fluid collection assembly 400. The passageways 462 may be formed using any suitable technique. In an example, the friction material 461 may be applied to the contact surface 460 in a manner that causes peaks and valleys to form and at least some of the valleys form the passageways 462. In an example, the friction material 461 may include one or more cutouts formed therein either before or after applying the friction material 461 to the contact surface 460. The cutouts may form the passageways 462. In an example, the friction material 461 may include a plurality of pores (e.g., interconnected pores) that form the passageways 462.

The friction material 461 illustrated in FIGS. 4A and 4B are in the form of dots. The friction material 461 are in the form of dots when at least some of the friction material 461 are completely laterally surrounded by the porous material 415. Optionally, some of the friction material 461 may be on the boundary between the fluid impermeable barrier 408 and the contact surface 460 and such friction material 461 are collectively completely laterally surrounded by the porous material 415 and the fluid impermeable barrier 408. Forming the friction material 461 as dots may allow for selective placement of the friction material 461, such as to avoid the urethral opening or increase the likelihood that the friction material 461 contacts skin. Further, forming the friction material 461 as dots may decrease the surface area of the contact surface 460 that the friction material 461 covers thereby decreasing the likelihood that the friction material 461 obstructs fluid flow or causes splashing.

The friction material 461 does not have to be arranged on the porous material 415 in the form of dots. For example, FIGS. 4C-4E are isometric views of different fluid collection assemblies that include the friction material disposed therein in different arrangements, according to different embodiments. Except as otherwise disclosed herein, the fluid collection assemblies illustrated in FIGS. 4C-4E are the same or substantially similar to any of the fluid collection assemblies disclosed herein.

Referring to FIG. 4C, the fluid collection assembly 400c includes a porous material 415c extending across at least one opening 416c and at least one securement body 406c. The securement body 406c includes at least one friction material 461d disposed on the porous material 415c. The friction material 461 is arranged as one or more longitudinally extending strips that extend generally parallel to a longitudinal axis of the fluid collection assembly 400c. The strips are more likely to contact anatomical features that extend obliquely or perpendicularly relative to a longitudinal axis of the of the fluid collection assembly 400c, such as the clitoral head, the mons pubis, and the perineum. The strips may also contact a greater percentage of anatomical features that generally extend parallel to the longitudinal axis of the fluid collection assembly 400c, such as the labia folds, if correctly positioned (e.g., the space between the strips are not spaced directly over the labia folds).

Referring to FIG. 4D, the fluid collection assembly 400d includes a porous material 415d extending across at least one opening 416d and at least one securement body 406d. The securement body 406d includes at least one friction material 461d disposed on the porous material 415d. The friction material 461d is arranged as one or more longitudinally extending strips that extend generally perpendicular to a longitudinal axis of the fluid collection assembly 400c. The strips are more likely to contact anatomical features that extend obliquely or parallel to a longitudinal axis of the of the fluid collection assembly 400c, such as the labia folds. For example, the strips illustrated in FIG. 4D are more likely to contact the labia folds than the strips illustrated in FIG. 4C regardless of the position of the fluid collection assemblies thereof. However, the strip illustrated in FIG. 4C may contact a greater percentage of the labia folds if the strips are disposed directly over the labia folds than the strips illustrated in FIG. 4D.

Referring to FIG. 4E, the fluid collection assembly 400e includes a porous material 415e extending across at least one opening 416e and at least one securement body 406e. The securement body 406e includes at least one friction material 461e disposed on the porous material 415e. The friction material 461e may be disposed on the porous material 415e to cover substantially all of the porous material. As such, the friction material 461e is more likely to contact skin and to contact a greater quantity of skin that the friction material illustrated in FIGS. 4A-4D. However, the friction material 461e must define one or more passageways 462e therethrough to allow the bodily fluids to flow into the chamber thereof (not shown).

It is noted that the arrangement of the friction material illustrated in FIGS. 4A-4E are provided for illustrative purposes only and that the friction material disclosed herein may exhibit different arrangements. In an example, the friction material may be arranged in strips that extend obliquely relatively to the longitudinal axis of the fluid collection assembly. In an example, the friction material may be arranged in the form of one or more dashed lines. In an example, the friction material may be arranged in a curved line. In an example, the friction material may be arranged in a combination of any of the arrangements disclosed herein (e.g., a checkered arrangement that includes a plurality of strips that extend parallel to and perpendicular to a longitudinal axis of the fluid collection assembly).

The friction materials disclosed above include the friction materials disposed on the porous material of female fluid collection assemblies. However, it is noted that the friction materials disclosed herein may be disposed on the porous materials of any of the male fluid collection assemblies disclose herein. Such friction materials may help maintain the penis in the chambers of such male fluid collection assemblies when the penis would otherwise become buried thereby preventing or minimizing pooling of bodily fluids caused by buried penises. The arrangement of the friction material on the porous materials of the male fluid collection assemblies may be the same or substantially similar to the arrangement of any of the friction material disclosed above with regards to the female fluid collection assemblies.

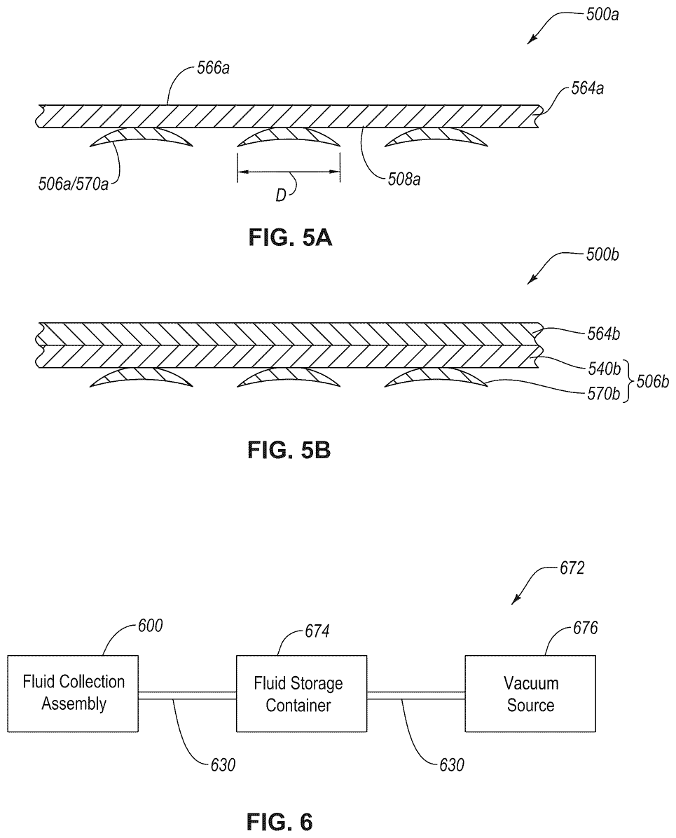

The fluid collection assemblies disclosed herein may include securement body instead of or in addition to the fibers and the friction materials disclosed above. For example, FIG. 5A is a cross-sectional schematic of a portion of a fluid collection assembly 500a, according to an embodiment. The fluid collection assembly 500a includes a base layer 564a. The base layer 564a may be, for example, a fluid impermeable layer, porous material, or a substrate of a base. The base layer 564a includes a top surface 566a and a bottom surface 568a. The bottom surface 568a is configured to be closer to a patient (e.g., adjacent to the skin of the patient) than the top surface 566a during use.

The fluid collection assembly 500a includes at least one securement body 506a. The securement body 506a include one or more suction cups 570a directly attached to or integrally formed with the base layer 564a. In particular, the suction cups 570a extend from the bottom surface 568a of the base layer 564a. The suction cups 570a are formed from a flexible material that is impermeable to air, such as nitrile, neoprene, polyurethane, silicone, rubber, vinyl, any other suitable polymer, or combinations thereof. The suction cups 570a may exhibit any concave shape. For example, the suction cups 570a may exhibit a conical or other cup-like shape having, for example, a circular or oblong opening.

The suction cups 570a may exhibit a maximum lateral dimension D (e.g., diameter). The maximum lateral dimension D of the suction cups 570a may be selected to be about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.2 cm, about 1 cm to about 1.4 cm, about 1.2 cm to about 1.6 cm, about 1.4 cm to about 1.8 cm, about 1.6 cm to about 2 cm, about 1.8 cm to about 2.25 cm, about 2 cm to about 2.5 cm, about 2.25 cm to about 2.75 cm, about 2.5 cm to about 3 cm, about 2.75 cm to about 3.5 cm, or about 3 cm to about 4 cm. The maximum lateral dimension D of the suction cups 570a may be selected based on a number of factors. Generally, the maximum lateral dimension D may be selected based on two competing factors, namely increasing the force required to detach the suction cups 570a and minimizing hickies and general discomfort caused by the suction cups 570a. For example, generally increasing the lateral dimension D may increase the force required to detach the suction cups 570a but also increases the likelihood of hickies and/or patient discomfort, especially if the fluid collection assembly 500a is used for a prolonged period of time. However, decreasing the maximum lateral dimension D may allow the fluid collection assembly 500a to include more suction cups 570a which may at least partially offset the decrease force required to detach the suction cups 570a.

FIG. 5B is a cross-sectional schematic of a portion of a fluid collection assembly 500b, according to an embodiment. Except as otherwise disclosed herein, the fluid collection assembly 500b is the same or substantially similar to the fluid collection assembly 500a. For example, the fluid collection assembly 500b may include a base layer 564b and at least one securement body 506b. However, the securement body 506b may include a support 540b and a plurality of suction cups 570b extending from the support 540b. The support 540b may the same or substantially similar to the support 140 illustrated in FIG. 1C. The support 540b may facilitate manufacture of the base layer 564b. For example, the base layer 564b may be manufactured using traditional methods (e.g., extrusion, tape casting, etc.) instead of other techniques that are required to form the base layer 564b with the suction cups 570b directly disposed therein.

FIG. 6 is a block diagram of a system 672 for fluid collection, according to an embodiment. The system 672 includes a fluid collection assembly 600, a fluid storage container 674, and a vacuum source 676. The fluid collection assembly 600, the fluid storage container 674, and the vacuum source 676 may be fluidly coupled to each other via one or more conduits 630. For example, fluid collection assembly 600 may be operably coupled to one or more of the fluid storage container 674 or the vacuum source 676 via the conduit 630. Bodily fluids (e.g., urine or other bodily fluids) collected in the fluid collection assembly 600 may be removed from the fluid collection assembly 600 via the conduit 630 which protrudes into the fluid collection assembly 600. For example, an inlet of the conduit 630 may extend into the fluid collection assembly 600, such as to a reservoir therein. The outlet of the conduit 630 may extend into the fluid storage container 674 or the vacuum source 676. Suction force may be introduced into the chamber of the fluid collection assembly 600 via the inlet of the conduit 630 responsive to suction (e.g., vacuum) force applied at the outlet of the conduit 630.

The suction force may be applied to the outlet of the conduit 630 by the vacuum source 676 either directly or indirectly. The suction force may be applied indirectly via the fluid storage container 674. For example, the outlet of the conduit 630 may be disposed within the fluid storage container 674 and an additional conduit 630 may extend from the fluid storage container 674 to the vacuum source 676. Accordingly, the vacuum source 676 may apply suction to the fluid collection assembly 600 via the fluid storage container 674. The suction force may be applied directly via the vacuum source 676. For example, the outlet of the conduit 630 may be disposed within the vacuum source 676. An additional conduit 630 may extend from the vacuum source 676 to a point outside of the fluid collection assembly 600, such as to the fluid storage container 674. In such examples, the vacuum source 676 may be disposed between the fluid collection assembly 600 and the fluid storage container 674.

The fluid collection assembly 600 may be similar or identical to any of the fluid collection assemblies disclosed herein in one or more aspects. The fluid collection assembly 600 may be shaped and sized to be positioned adjacent to a female urethral opening or have a male urethral opening positioned therethrough (e.g., receive a penis therein). For example, the fluid collection assembly 600 may include a fluid impermeable barrier at least partially defining a chamber (e.g., interior region) of the fluid collection assembly 600. The fluid impermeable barrier also defines at least one opening extending therethrough from the external environment. The opening may be positioned adjacent to a female urethral opening or have a male urethral opening positioned therethrough. The fluid collection assembly 600 may include porous material disposed in the chamber such as one or more of a fluid permeable support and a fluid permeable membrane. The fluid collection assembly 600 includes one or more of any of the securement bodies disclosed herein.

The fluid storage container 674 is sized and shaped to retain the bodily fluids therein. The fluid storage container 674 may include a bag (e.g., drainage bag), a bottle or cup (e.g., collection jar), or any other enclosed container for storing bodily fluids such as urine. In some examples, the conduit 630 may extend from the fluid collection assembly 600 and attach to the fluid storage container 674 at a first point therein. An additional conduit 630 may attach to the fluid storage container 674 at a second point thereon and may extend and attach to the vacuum source 676. Accordingly, a vacuum (e.g., suction) may be drawn through fluid collection assembly 600 via the fluid storage container 674. Fluid, such as urine, may be drained from the fluid collection assembly 600 using the vacuum source 676.

The vacuum source 676 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 676 may provide a vacuum or suction to remove fluid from the fluid collection assembly 600. In some examples, the vacuum source 676 may be powered by one or more of a power cord (e.g., connected to a power socket), one or more batteries, or even manual power (e.g., a hand operated vacuum pump). In some examples, the vacuum source 676 may be sized and shaped to fit outside of, on, or within the fluid collection assembly 600. For example, the vacuum source 676 may include one or more miniaturized pumps or one or more micro pumps. The vacuum sources 676 disclosed herein may include one or more of a switch, a button, a plug, a remote, or any other device suitable to activate the vacuum source 676.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier at least defining a chamber, at least one opening and a fluid outlet;
at least one porous material disposed in the chamber, the at least one porous material includes at least one contact surface that is configured to contact a patient during use; and
at least one friction material disposed on at least a portion of the at least one contact surface, the at least one friction material exhibiting a coefficient of static friction that is greater than the at least one contact surface.

3. The fluid collection assembly of claim 2, wherein the at least one friction material is different than the at least one porous material forming the at least one contact surface.

4. The fluid collection assembly of claim 2, wherein the at least one friction material includes at least one elastomer.

5. The fluid collection assembly of claim 4, wherein the at least one elastomer includes silicone.

6. The fluid collection assembly of claim 4, wherein the at least one elastomer includes at least one of nitrile, rubber, or neoprene.

7. The fluid collection assembly of claim 2, wherein the at least one friction material is attached to the at least one porous material using an adhesive.

8. The fluid collection assembly of claim 2, wherein the at least one friction material extends into some pores of the at least one porous material.

9. The fluid collection assembly of claim 2, wherein the at least one friction material defines one or more passageways extending therethrough.

10. The fluid collection assembly of claim 9, wherein the at least one friction material is a porous material defining a plurality of pores, the plurality of pores forming the one or more passageways extending through the at least one friction material.

11. The fluid collection assembly of claim 9, wherein the one or more passageways include one or more cutouts extending through the at least one friction material.

12. The fluid collection assembly of claim 9, wherein the at least one friction material is disposed on substantially all of the at least one contact surface.

13. The fluid collection assembly of claim 2, wherein the at least one friction material includes a plurality of friction materials disposed on the at least one porous material, each of the plurality of friction materials are spaced from each other.

14. The fluid collection assembly of claim 13, wherein the plurality of friction materials includes a plurality of dots of the at least one friction material.

15. The fluid collection assembly of claim 13, wherein the plurality of friction materials includes a plurality of longitudinally extending strips that are generally parallel to a longitudinal axis of the fluid collection assembly.

16. The fluid collection assembly of claim 13, wherein the plurality of friction materials includes a plurality of transversely extending strips that are generally perpendicular to a longitudinal axis of the fluid collection assembly.

17. The fluid collection assembly of claim 2, wherein the at least one friction material is spaced from a portion of the at least one contact surface that is configured to abut or be positioned adjacent to a urethra opening.

18. The fluid collection assembly of claim 2, wherein:
the at least one opening includes an elongated opening configured to be positioned adjacent to female anatomy; and
a portion of the at least one porous material extends across the elongated opening, the contact surface forming a portion of the at least one porous material extending across the elongated opening.

19. The fluid collection assembly of claim 2, wherein the at least one opening is configured to receive male anatomy.

20. The fluid collection assembly of claim 2, further comprising at least one of:
a plurality of fibers and a plurality of branches extending from at least some of the plurality of fibers, the plurality of fibers exhibiting an average lateral dimension of about 5 μm or less, the plurality of branches exhibiting an average diameter of 300 nm to 2 μm, one or more of the plurality of branches extending from or attached to a portion of one of the plurality of fibers that is spaced from a terminal end of the one of the plurality of fibers, wherein the terminal end of the one of the plurality of fibers is an end of the one of the plurality of fibers that is farthest spaced from the at least one exterior surface; or
a plurality of suction cups.

21. A fluid collection system, comprising:
a fluid storage container configured to hole one or more bodily fluids therein;
a fluid collection assembly including:
a fluid impermeable barrier at least defining a chamber, at least one opening and a fluid outlet;
at least one porous material disposed in the chamber, the at least one porous material includes at least one contact surface that is configured to contact a patient during use; and
at least one friction material disposed on at least a portion of the at least one contact surface, the at least one friction material exhibiting a coefficient of static friction that is greater than the at least one contact surface; and
a vacuum source in fluid communication with the fluid storage container and the fluid collection assembly, the vacuum source configured to draw the one or more bodily fluids from the fluid collection assembly and deposit the one or more bodily fluids in the fluid storage container via one or more conduits.
